(19) United States
(12) Patent Application Publication
(10) Pub. No.: US 2012/0122997 A1
(43) Pub. Date: May 17, 2012

(54) NEW POLYSACCHARIDE-BASED MATERIALS

(76) Inventors: Andrew P. Abbott, Leicester (GB); Andrew Ballantyne, Leicester (GB)

(21) Appl. No.: 13/381,007

(22) PCT Filed: Jun. 29, 2010
(86) PCT No.: PCT/GB2010/001262

§ 371 Date: Feb. 1, 2012

(30) Foreign Application Priority Data
Jun. 29, 2009 (GB) 0911172.5

(51) Int. Cl. C08L 3/00; C09K 3/18; A23L 1/058; H01B 1/12; A23L 1/27; C08L 1/00; A61K 47/36

(52) U.S. Cl. 514/778; 106/214.2; 106/163.01; 106/2; 524/52; 514/777; 252/500; 426/540; 426/573; 426/658

(57) ABSTRACT

There is provided a plastic or gel material comprising a mixture of: (a) a compound of formula (I) or a mixture of two or more compounds of formula (I), (R⁻)ₙ(Xⁿ⁻) (I) or a hydrate thereof, wherein n, R⁺ and Xⁿ⁻ have meanings given in the description; (b) one or more uncharged organic compounds, each of which compounds comprises at least one oxygen atom and at least one hydrogen atom that is capable of forming a hydrogen bond with Xⁿ⁻; and (c) one or more polysaccharides, wherein each polysaccharide is a polymer of pyranose monomers, at least 30% of which monomers are in the α-anomeric conformation. There is also provided articles formed from such materials, uses of such materials and processes for forming such materials.

This invention relates to novel plastic or gel materials based upon certain polysaccharides (pyranose-based polysaccharides containing at least 30% of the pyranose monomers in the α-anomeric form). This invention also relates to uses of such materials, processes for their preparation as well as to articles comprising such materials.

The listing or discussion of a prior-published document in this specification should not necessarily be taken as an acknowledgment that the document is part of the state of the art or is common general knowledge.

There are numerous known plastic materials. Due to their ability to be moulded or manipulated into almost any given shape, such materials find an immense variety of uses in day-to-day life. For example, moulded plastics can be used as (structural components of) utensils or other articles, foamed plastics can be used as packaging materials, and plastics in the form of a film can be used for food coverings or as wrapping/packaging for food or other items.

The properties of plastic materials derive from polymer chains that are present in those materials. Many polymers employed in current plastic materials are ultimately derived from fossil fuels and/or are not biodegradable/compostable.

There are a number of known plastic materials that are based upon natural polymers (or polymers that are otherwise biodegradable), such as the plastic materials disclosed in U.S. Pat. No. 5,665,786 and US 2004/0249065 A1, as well as in the documents discussed in the background sections of those documents. Although these materials have found various applications, their usefulness has been restricted by the inherent limitations imposed by the mechanical and/or physicochemical properties of the polymers that they contain, or by the processes by which the materials are made.

Thus, there are still many fossil fuel-derived plastic materials that, due to the (advantageous) mechanical and/or physicochemical properties that they possess, either:
(i) have not yet been (entirely) supplanted by biodegradable equivalents; or
(ii) do not have a biodegradable equivalent.

In the light of the above, it can be seen that there remains a need for further plastic materials based upon natural or biodegradable polymers. A similar need exists for gels based upon such polymers.

Natural polymers are diverse in nature and encompass groups of materials such as proteins, polynucleotides (e.g. DNA and RNA) and polysaccharides. For many years, attempts have been made to manipulate such natural polymers to form plastic materials. For example, in 1855, the material Parkesine was developed. This material derives from the manipulation of a natural polysaccharide (cellulose) through treatment with nitric acid and a solvent, followed by dissolution in alcohol and subsequent hardening.

However, despite some successes, the inherent mechanical and physicochemical properties of natural polysaccharides have so far limited their use in the production of plastic materials (especially plastic materials having high flexibility).

It is known that cellulose can be dissolved in high ionic strength liquids, such as liquid (molten) quaternary ammonium salts (see, for example Zhu et al., Green Chem. 2006, 8, 325 as well as international patent application WO 03/029329) or mixtures of quaternary ammonium salts and nitrogen-containing bases (see, for example U.S. Pat. No. 1,943,176). The dissolution of cellulose in such liquids facilitates the processing of that polysaccharide into different physical forms, which is achieved through precipitation of the cellulose from solution (e.g. by addition of water or an alcohol). However, the formation of a plastic or gel material from a pyranose polysaccharide containing at least 30% of the pyranose monomers in the α-anomeric form, an ammonium or phosphonium salt and an uncharged organic compound which is capable of forming a hydrogen bond has neither been disclosed nor suggested.

Polymeric materials containing starch and plasticisers such as polyols are known (see, for example, WO 2008/071717 and WO 2008/090195). However, such materials do not contain salts of ammonium or phosphonium ions. Further, these known materials require the presence of polymers other than polysaccharides to achieve acceptable mechanical properties.

We have now found, surprisingly, that a plastic or gel material may be formed from a polysaccharide that is a polymer of pyranose monomers, at least 30% of which monomers are in the α-anomeric conformation, through mixing that polysaccharide with an uncharged, organic H-bond donor/acceptor and a salt of an ammonium or phosphonium ion.

Thus, according to a first aspect of the invention there is provided a plastic or gel material comprising a mixture of:
(a) a compound of formula (I) or a mixture of two or more compounds of formula (I),

(R⁺)ₙ(Xⁿ⁻) (I)
or a hydrate thereof, wherein
n is 1, 2 or 3,
R⁺ is a primary, secondary, tertiary, quaternary or unsubstituted ammonium cation or a quaternary phosphonium cation,
Xⁿ⁻ is a monovalent, bivalent or trivalent anion;
(b) one or more uncharged organic compounds, each of which compounds comprises at least one oxygen atom and at least one hydrogen atom that is capable of forming a hydrogen bond with Xⁿ⁻; and
(c) one or more polysaccharides, wherein each polysaccharide is a polymer of pyranose monomers, at least 30% of which monomers are in the α-anomeric conformation.

When used herein, the term “plastic material” refers to a material that can be moulded (i.e. a material that has the property of plasticity).

In certain embodiments of the first aspect of the invention, the plastic material is flexible and/or ductile.

Flexibility of the plastic material can be determined, for example, by measurement of deformation angle tolerance (e.g. by taking a standard size of sample, such as a film sample from 0.1 and 0.5 mm thick, 2.54 cm wide and 15 cm long, placing the sample on a horizontal surface and raising one half of the length of the sample while keeping the other half stationary, and then determining, e.g. at a temperature of 298 K, the maximum angle between the raised half of the sample and the horizontal surface that can be formed without breaking the specimen).

Embodiments of the first aspect of the invention relating to the plastic material include those in which the deformation angle tolerance (e.g. when measured at a temperature of 298 K) is at least 5°, such as at least 10°, 15°, 20°, 25°, 30°, 35°, 40° or 45°.

As will be appreciated by those skilled in the art, the term “ductile” refers to the ability of a material to be deformed plastically without fracture. Ductility of the plastic material can be determined, for example, by measurement of elongation to break. This can be done, for example, by taking a standard size of sample, such as a 10 mm diameter cylindrical sample between 3 and 5 cm long, securing one end of the sample to a fixed anchor and the distal end of the sample to a mobile anchor, applying force to the mobile anchor so as to move it away from the fixed anchor (thereby applying strain along the length of the sample) and then measuring the force required to extend the sample as well as the percentage elongation of the sample at fracture. By way of such measurements, not only can information be obtained on elongation to break of the plastic material, but also on the tensile strength (including the Ultimate Tensile Strength) of that material.

Embodiments of the first aspect of the invention relating to the plastic material include those in which the elongation to break of the plastic material (e.g. when measured at a temperature of 298 K) is at least 5%, such as 10%, 15%, 25%, 30%, 35% or, particularly, 40%. Further embodiments of the first aspect of the invention relating to the plastic material include those in which the Ultimate Tensile Strength of the plastic material (e.g. when measured at a temperature of 298 K) is from 10 to 10,000 kN/m² (e.g. from 10 to 1000 kN/m²), such as from 20 to 700 kN/m² or, particularly, from 30 to 450 kN/m².

The term “gel material” will be well understood by those skilled in the art. As used herein, the term “gel” takes its normal meaning, and therefore includes references to substantially dilute crosslinked systems that exhibit no flow.

Component (a)

In certain embodiments of the first aspect of the invention, component (a) is a mixture of two or more compounds of formula (I) or, particularly, a compound of formula (I). In such embodiments, R⁺ may be a quaternary phosphonium cation or, particularly, a primary, secondary, tertiary, quaternary or unsubstituted ammonium cation (e.g. a cation R⁺ as defined in respect of formula (Ia) below).

Anion Xⁿ⁻ is any monovalent, bivalent or trivalent anion. Embodiments of the invention include those in which Xⁿ⁻ is an anion selected from the list comprising halide, chlorate, perchlorate, bromate, nitrate, nitrite, cyanide, cyanate, thiocyanate, hydrogencarbonate, carbonate, sulfate, hydrogensulfate, pyrosulfate, sulfite, hydrogensulfite, phosphate, monohydrogenphosphate, dihydrogenphosphate, metaphosphate, pyrophosphate, hexafluorophosphate, tetrafluoroborate, borate, diborate, triborate, tetraborate, carboxylate (e.g. any one of formate, acetate, trifluoroacetate, propionate, isobutyrate, heptanoate, decanoate, caprate, caprylate, stearate, acrylate, caproate, propiolate, ascorbate, citrate, glucuronate, glutamate, glycolate, α-hydroxybutyrate, lactate, tartrate, phenylacetate, mandelate, phenylpropionate, phenylbutyrate, benzoate, chlorobenzoate, methylbenzoate, hydroxybenzoate, methoxybenzoate, dinitrobenzoate, o-acetoxybenzoate, salicylate, nicotinate, isonicotinate, cinnamate, oxalate, malonate, succinate, suberate, sebacate, fumarate, malate, maleate, hydroxymaleate, hippurate, phthalate, terephthalate and the like) and sulfonate (e.g. any one of benzenesulfonate, methyl-, bromo- or chloro-benzenesulfonate, xylenesulfonate, methanesulfonate, trifluoromethanesulfonate, ethanesulfonate, propanesulfonate, hydroxyethanesulfonate, 1- or 2-naphthalene-sulfonate, 1,5-naphthalenedisulfonate and the like).

Particular embodiments of the invention that may be mentioned include those in which Xⁿ⁻ is a monovalent anion (i.e. where n is 1), such as a monovalent anion selected from the list above (e.g. an anion selected from the list comprising fluoride, chloride, bromide, iodide, nitrate and acetate (such as bromide or, particularly, chloride)).

When Xⁿ⁻ is a monovalent anion, the compound of formula (I) may be a compound of formula (Ia),

(R⁺)(X⁻) (Ia)

wherein:

R represents Het substituted by R⁵, or R represents a structure represented by formula (II)

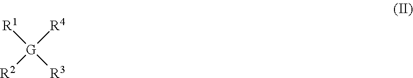

in which G represents N or P;

X⁻ is a monovalent anion;

R¹ to R⁵ independently represent
phenyl, C₁₋₁₀ alkyl, C₂₋₁₀ alkenyl or C₂₋₁₀ alkynyl, which latter four groups are optionally substituted by one or more R⁶ groups, or
C₃₋₁₀ cycloalkyl or C₄₋₁₀ cycloalkenyl which latter four groups are optionally substituted by one or more substituents selected from R⁶ and oxo,
or R⁵ represents H or, when G represents N, R¹ to R⁴ may also independently represent H;

Het represents a 4- to 14-membered heterocyclic group containing a N-atom that is bonded to the substituent R⁵, which heterocyclic group
(i) optionally contains one or more further heteroatoms selected from O, N and/or S,
(ii) may be fully saturated, partially unsaturated or aromatic,
(iii) may comprise one, two or three rings and
(iv) may be substituted by one or more further substituents selected from R⁶ and oxo;

R⁶ represents, independently at each occurrence,
halo,
CN,
C₁₋₁₀ alkyl, C₂₋₁₀ alkenyl, C₂₋₁₀ alkynyl, C₃₋₁₀ cycloalkyl, C₄₋₁₀ cycloalkenyl, which latter five groups are optionally substituted by one or more substituents selected from halo, C₁₋₄ alkyl, phenyl (optionally substituted by one or more substituents selected from OH, halo, C₁₋₄ alkyl and C₁₋₄ alkoxy) and OR^{6a},
OR^{6b},
SR^{6a},
N(R^{6d})(R^{6e}),
phenyl, which latter group is optionally substituted by one or more substituents selected from OH, halo, C₁₋₄ alkyl and C₁₋₄ alkoxy, or
B¹-A-B²—R^{6f};

R^{6a} to R^{6f} independently represent, at each occurrence,
H,
C₁₋₁₀ alkyl, C₂₋₁₀ alkenyl, C₂₋₁₀ alkynyl, C₃₋₁₀ cycloalkyl, C₄₋₁₀ cycloalkenyl, which latter five groups are optionally substituted by one or more substituents selected from halo, C₁₋₄ alkyl, phenyl (optionally substituted by one or more substituents selected from OH, halo, C₁₋₄ alkyl and C₁₋₄ alkoxy), OH and C₁₋₄ alkoxy, or
phenyl (optionally substituted by one or more substituents selected from OH, halo, C₁₋₄ alkyl and C₁₋₄ alkoxy);

A represents C(O) or S(O)₂;

B¹ and B² independently represent a direct bond, O, S, or N(R⁷); and

R⁷ represents H, C₁₋₄ alkyl, C₃₋₇ cycloalkyl or phenyl, which latter group is optionally substituted by one or more substituents selected from OH, halo, C₁₋₄ alkyl and C₁₋₄ alkoxy.

Unless otherwise specified, alkyl groups as defined herein may be straight-chain or, when there is a sufficient number (i.e. a minimum of three) of carbon atoms be branched-chain.

Het groups are 4- to 14-membered heterocyclic groups containing a N-atom that is bonded to the substituent R⁵. Values of heterocyclic Het groups that may be mentioned include 1-azabicyclo-[2.2.2]octanyl, benzimidazolyl, benzo[c]isoxazolidinyl, benzisoxazolyl, benzofurazanyl, benzomorpholinyl, 2,1,3-benzoxadiazolyl, benzoxazolidinyl, benzoxazolyl, benzopyrazolyl, benzo[e]pyrimidine, 2,1,3-benzothiadiazolyl, benzothiazolyl, benzotriazolyl, cinnolinyl, 2,3-dihydrobenzimidazolyl, 1,3-dihydro-2,1-benzisoxazolyl, 2,3-dihydro-pyrrolo[2,3-b]pyridinyl, hexahydropyrimidinyl, hydantoinyl, imidazolyl, imidazo[1,2-a]pyridinyl, imidazo[2,3-b]thiazolyl, indolyl, isoquinolinyl, isoxazolidinyl, isoxazolyl, maleimido, morpholinyl, oxadiazolyl, 1,2- or 1,3-oxazinanyl, oxazolyl, phthalazinyl, piperazinyl, piperidinyl, purinyl, pyrazinyl, pyrazolyl, pyridazinyl, pyridinyl, pyrimidinyl, pyrrolidinonyl, pyrrolidinyl, pyrrolinyl, pyrrolo[2,3-b]pyridinyl, pyrrolo[5,1-b]pyridinyl, pyrrolo[2,3-c]pyridinyl, pyrrolyl, quinazolinyl, quinolinyl, 4,5,6,7-tetrahydrobenzimidazolyl, 4,5,6,7-tetrahydrobenzopyrazolyl, 5,6,7,8-tetrahydrobenzo[e]pyrimidine, 3,4,5,6-tetrahydropyridinyl, 1,2,3,4-tetrahydropyrimidinyl, 3,4,5,6-tetrahydropyrimidinyl, thiadiazolyl, thiazolidinyl, thiazolyl, thieno[5,1-c]pyridinyl, triazolyl, 1,3,4-triazolo[2,3-b]pyrimidinyl, and the like.

Particular values of Het that may be mentioned include triazolyl and, particularly, imidazolyl.

Embodiments of the first aspect of the invention that may be mentioned include those in which:
(1) X⁻ is a halide, nitrate or carboxylate anion (such as acetate, nitrate, bromide or, particularly, chloride);
(2) G represents N;
(3) R¹ to R³ independently represent C₁₋₈ alkyl (or, particularly, C₁₋₆ alkyl) optionally substituted by one R⁶ group;
(4) R⁴ and R⁵ independently represent C₁₋₈ alkyl or C₂₋₈ alkenyl (e.g. C₁₋₆ alkyl or C₂₋₆ alkenyl), which latter two groups are optionally substituted by one to three substituents selected from halo, phenyl (optionally substituted by one or more substituents selected from halo, C₁₋₄ alkyl and C₁₋₄ alkoxy) and C₁₋₄ alkoxy;
(5) when G represents N, R¹ to R⁴ may alternatively, and independently, represent H;
(6) R⁶ represents, halo, OR^{6b}, phenyl (which latter group is optionally substituted by one or more substituents selected from halo, C₁₋₄ alkyl and C₁₋₄ alkoxy) or B¹-A-B²−R^{6f};
(7) R^{6b} represents H or C₁₋₄ alkyl;
(8) R^{6f} represents H, C₁₋₄ alkyl or phenyl;
(9) A represents C(O);
(10) B¹ represents S or, particularly O;
(11) B² represents a direct bond;
(12) Het¹ represents a 5- or 6-membered monocyclic, aromatic heterocyclic group containing a N-atom that is bonded to the substituent R⁵, which heterocyclic group
   (i) optionally contains one to three further N-atoms, and
   (ii) may be substituted by one or two further substituents selected from halo, C₁₋₆ alkyl or C₂₋₆ alkenyl, which latter two groups are optionally substituted by one to three substituents selected from halo, phenyl (optionally substituted by one or more substituents selected from halo, C₁₋₄ alkyl and C₁₋₄ alkoxy) and C₁₋₄ alkoxy.

Further embodiments of the invention include those in which:
(1) R¹ to R³ independently represent C₁₋₄ alkyl or, particularly, C₁₋₂ alkyl;
(2) R⁴ represents C₁₋₄ alkyl (e.g. C₁₋₂ alkyl) optionally substituted by phenyl, OH or —O—C(O)—(C₁₋₂ alkyl);
(3) R⁵ represents C₁₋₅ alkyl (e.g. C₂₋₅ alkyl, such as C₂ or C₄ alkyl) optionally substituted by phenyl (e.g. R⁵ represents unsubstituted C₁₋₅ alkyl (e.g. C₂₋₅ alkyl, such as C₂ or C₄ alkyl));
(4) Het represents a 5-membered monocyclic, aromatic heterocyclic group containing a N-atom that is bonded to the substituent R⁵, which heterocyclic group
   (i) contains one or two further N-atoms, and
   (ii) may be substituted by a further substituent that is a C₁₋₆ alkyl group.

Still further embodiments of the invention include those in which Het-R⁵ represents a triazole or imidazole substituted by R⁵ and optionally further substituted by C₁₋₆ alkyl (e.g. C₁₋₄ alkyl or, particularly, C₁₋₂ alkyl).

Thus, embodiments of the invention include those in which Het-R⁵ represents the following structure

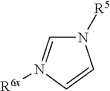

in which
R⁵ is as defined above, but particularly represents unsubstituted C₂₋₅ alkyl, such as C₂ or C₄ alkyl (e.g. ethyl or n-butyl); and
R^{6x} represents C₁₋₆ alkyl (e.g. C₁₋₄ alkyl, such as ethyl or, particularly, methyl).

Still further embodiments of the invention include those in which the compound of formula (Ia) is represented by the following structure

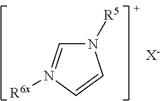

in which R⁵, R^{6x} and X⁻ are as defined above (for example: R⁵ represents unsubstituted C₂₋₅ alkyl, such as C₂ or C₄ alkyl (e.g. ethyl or n-butyl); R^{6x} represents C₁₋₆ alkyl (e.g. C₁₋₄ alkyl, such as ethyl or, particularly, methyl); X⁻ represents bromide or, particularly, chloride).

Particular embodiments of the first aspect of the invention that may be mentioned include those in which the compound of formula (I) is one in which G represents N and the compound of formula (I) is therefore an ammonium salt or a primary, secondary, tertiary or, particularly, quaternary ammonium salt. In these embodiments, the anion(s) present in the salt(s) may be halide ions.

In this respect, quaternary ammonium salts that may be mentioned include those selected from the list comprising benzyltrimethylammonium halide, tetrabutylammonium halide, ethylmethylimidazolium halide, acetylcholine halide and choline halide (wherein halide is fluoride, iodide, bromide or, particularly, chloride).

Component (b)

The term “organic” will be well understood by those skilled in the art. Thus, when used herein, the term “organic” includes references to uncharged chemical compounds (other than carbon, oxides of carbon, or acids of (bi)carbonate, cyanide, cyanate, thiocyanate or fulminate), whose molecules contain carbon.

The term “uncharged”, when used herein in relation to component (b), refers to organic molecules (compounds) that do not bear a permanent positive or negative (electrostatic) charge on any atom within the molecule. In this respect, uncharged organic compounds are those that comprise a single, covalently-bonded molecule and that are not separated into cationic and anionic components.

Whether a compound contains a hydrogen atom that is capable of forming a hydrogen bond with Xⁿ⁻ will either be evident to those skilled or can be determined by methods known to those skilled in the art (see, for example, Paul D. Beer, Philip A. Gale and David K. Smith, Supramolecular Chemistry (Oxford Chemistry Primers), Oxford University Press, Oxford, 1999, and especially Chapter 3 (pages 31 to 42) and the references cited therein). For example, a titration can be conducted in which anion Xⁿ⁻ is added to the dissolved compound and changes in a physical property connected with the H-atoms of the compound (e.g. a spectroscopic signal, such as an infrared or ¹H n.m.r. signal) are monitored. For instance, the compound in question may be dissolved in a deuterated solvent (such as deuterated chloroform, dichloromethane or acetonitrile) and changes in the ¹H n.m.r. signals from that compound monitored when aliquots of a quaternary ammonium salt (such as a tetrabutylammonium salt) having Xⁿ⁻ as anion are added to the solution.

Embodiments of the first aspect of the invention also include those in which the or each compound forming component (b) has:
(i) a melting point greater than −20° C. (e.g. from −20 to 200, 180, 160 or, particularly, 140° C.); and/or
(ii) a molecular weight of less than 200 g/mol (e.g. from 45 to 200, 180, 160, 140 or, particularly, 120 g/mol).

In these and other embodiments of the first aspect of the invention, the or each compound forming component (b) may be an amide or polyol.

When used herein in connection with component (b), the term “amide” includes references to compounds containing a —C(O)N(H)— structural fragment. Further when used herein in connection with component (b), the term “polyol” includes references to compounds containing two or more hydroxyl (—OH) groups.

Thus, embodiments of the first aspect of the invention include those in which component (b) is a one or more compounds of formula (IIIa) and/or one or more compounds of formula (IIIb),

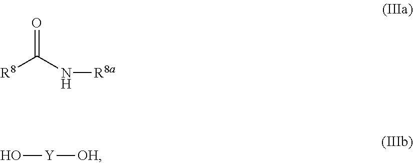

wherein:

R⁸ represents H, C₁₋₄ alkyl (which latter group is optionally substituted by one or more F atoms), phenyl (which latter group is optionally substituted by one or more substituents selected from halo, C₁₋₄ alkyl and C₁₋₄ alkoxy) or N(R⁹)R¹⁰;

R^{8a} represents H or C₁₋₄ alkyl (which latter group is optionally substituted by one or more F atoms);

R⁹ and R¹⁰ independently represent H or C₁₋₄ alkyl (which latter group is optionally substituted by one or more F atoms);

Y represents C₂₋₁₀ alkylene or C₄₋₈ cycloalkylene optionally
(i) substituted by one or more substituents selected from F, OH, SH, N(R¹¹)R¹² and C₁₋₄ alkyl (which latter group is optionally substituted by one or more substituents selected from F and OH), and/or
(ii) interrupted by one or more groups selected from O, S and NR¹³; and

R¹¹ to R¹³ independently represent H or C₁₋₄ alkyl (which latter group is optionally substituted by one or more substituents selected from F and OH).

Embodiments of the first aspect of the invention that may be mentioned include those in which:
(1) R⁸ represents H, CF₃, or, particularly, methyl, phenyl, NH₂, N(H)CH₃ or N(CH₃)₂;
(2) R^{8a} represents methyl or, particularly, H;
(3) A represents
   C₂₋₆ alkylene (e.g. C₂₋₆ n-alkylene) optionally substituted by or more (e.g. one, two, three or four) OH groups, or
   C₅₋₆ cycloalkylene substituted by one or more (e.g. five or six) substituents selected from OH and C₁₋₂ alkyl (which latter group is optionally substituted by two OH groups or, particularly, one OH group).

Further embodiments of the first aspect of the invention that may be mentioned include those in which:
(1) R⁸ represents methyl, phenyl, N(H)CH₃ or, particularly, NH₂;
(2) R^{8a} represents methyl or, particularly, H;
(3) A represents unsubstituted C₂ alkylene or C₃₋₆ n-alkylene optionally substituted by or more (e.g. one, two, three or four OH groups).

In certain embodiments of the first aspect of the invention, the compound of formula (IIIb) is a compound of formula (IV),

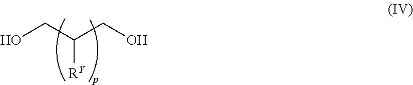

wherein:

R^{Y} is H or OH; and

p is 0 to 4 (e.g. 1, 2, 3 or 4).

Thus, particular embodiments of the first aspect of the invention that may be mentioned include those in which component (b) is one or more compounds selected from the list comprising benzamide, acetamide, N-methylurea, N,N′-dimethylurea urea, glycerol, mannitol, xylitol, ethylene glycol and propylene glycol (e.g. one or more compounds selected from the list comprising benzamide, acetamide, N-methylurea, N,N′-dimethylurea urea, glycerol, mannitol, xylitol and propylene glycol), or, alternatively, one or more compounds selected from the list comprising acetamide, glycerol or, particularly, urea.

Component (c)

As detailed above, component (c) is one or more polysaccharides, wherein each polysaccharide is a polymer of pyranose monomers, at least 30% of which monomers are in the α-anomeric conformation.

A pyranose monomer is a monomer of a pyranose polysaccharide that is based upon a (tetrahydro)pyran ring.

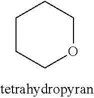

In polysaccharides, pyranose monomers are linked together by the formation of ether bonds involving an —OH group attached to a C-atom that is also attached to the O-atom of the (tetrahydro)pyran ring. This —OH group can be present in the cyclic monomer group in one of two conformations, namely the α- and the β-anomeric conformations (illustrated below by use of a particular “chair” conformation of the pyranose ring).

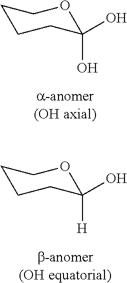

The C-atom in the above-depicted structures to which the two O-atoms are attached is called the anomeric carbon, and also represents a chiral centre when the molecule is locked in the ring conformation. In this respect, it is to be noted that the formation of the ring is reversible in aqueous solution for pyranose monomers, due to interconversion of the molecules between linear (hydroxyaldehyde) and cyclic (hemiacetal) forms.

The polysaccharides employed as component (c) in the first aspect of the present invention contain pyranose monomeric units, at least 30% of which monomers are in the α-anomeric conformation. The α-anomeric conformation in a polysaccharide is illustrated below by reference to the structure of amylose (which is used as an illustrative example only).

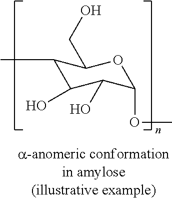

In amylose, the ether bonds are formed between the 1- and 4-positions of pyranose monomer (i.e. between the anomeric carbon and the C-atom in the 4-position in the ring relative to that carbon). Such linkages are described as α(1→4). However, the polysaccharides employed in the first aspect of the invention may contain any ether linkages found in polysaccharides derived from natural sources, such as α(1→6), β(1→4) and/or β(1→6), provided that at least 30% of the pyranose monomers are present in the α-anomeric conformation.

Embodiments of the first aspect of the invention that may be mentioned include those in which the or each polysaccharide of component (c) comprises:
one or more of amylose, amylopectin, agarose and agaropectin;
a mixture of amylose and amylopectin; or
a mixture of agarose and agaropectin.

Particular embodiments that may be mentioned in this respect include those in which component (c) is:
one or more polysaccharides (e.g. one polysaccharide) selected from the list comprising agar and a starch; or
one or more polysaccharides (e.g. one polysaccharide) selected from the list comprising agar and a starch selected from the list comprising corn starch, potato starch, wheat starch, tapioca starch and soluble starch.

Embodiments of the first aspect of the invention that may be mentioned include those in which the one or more polysaccharides of component (c) each comprise a minimum of 30 (e.g. a minimum of 40, 50, 75, 100, 200, 300, 500, 1000, 10000 or 20000) pyranose monomer units.

Further embodiments of the first aspect of the invention that may be mentioned include those in which the weight average molecular weight of the or each of the one or more polysaccharides of component (c) is at least 4 kDa (e.g. at least 6, 8, 12, 16, 32, 48, 60, 100, 300 or, particularly, 500 kDa).

Particular embodiments of the first aspect of the invention that may be mentioned include those in which:
polysaccharide component (c) is the sole polymeric component of the plastic or gel material; or
the plastic or gel material is substantially free of polymers that are not polysaccharides (or that are not polysaccharides that are polymers of pyranose monomers, at least 30% of which monomers are in the α-anomeric conformation).

The term “substantially free”, when used herein in relation to certain polymers, includes references to plastic or gel materials according to the first aspect of the invention that comprise at most 10% (e.g. at most 8, 6, 5, 4, 3, 2, 1 or 0.5%) by weight of polymers that are not polysaccharides (or that are not polysaccharides that are polymers of pyranose monomers, at least 30% of which monomers are in the α-anomeric conformation).

Other embodiments of the first aspect of the invention that may be mentioned include those in which the plastic or gel material is substantially free of water.

The term “substantially free”, when used herein in relation to water, includes references to plastic or gel materials according to the first aspect of the invention that comprise at most 10% (e.g. at most 8, 6, 5, 4, 3, 2, 1 or 0.5%) by weight of water.

For the avoidance of doubt, the first aspect of the invention encompasses embodiments wherein the gel or plastic material contains components described through a combination of any two or more embodiments of the first aspect of the invention that are detailed above and that are not mutually exclusive. Thus, for example a particular embodiment of the first aspect of the invention is a plastic or gel material comprising a mixture of:
(a) one or more compounds selected from the list comprising an ammonium halide salt or a primary, secondary, tertiary or quaternary ammonium halide salt;
(b) one or more compounds selected from the list comprising an amide and a polyol; and
(c) one or more polysaccharides, the or each polysaccharide comprising one or more of amylose, amylopectin, agarose and agaropectin.

Further, another particular embodiment of the first aspect of the invention is a plastic or gel material comprising a mixture of:
(a) one or more compounds (e.g. one compound) selected from the list comprising benzyltrimethylammonium halide, tetrabutylammonium halide, ethylmethylimidazolium halide, acetylcholine halide and choline halide (wherein halide is fluoride, iodide, bromide or, particularly, chloride);
(b) one or more compounds (e.g. one compound) selected from the list comprising benzamide, acetamide, N-methylurea, N,N′-dimethylurea, urea, glycerol, mannitol, xylitol and propylene glycol; and
(c) one or more polysaccharides (e.g. one polysaccharide) selected from the list comprising agar and a starch.

In still further embodiments of the first aspect of the invention, which further embodiments relate to all of the above-described embodiments, the plastic or gel material is one in which:
the molar ratio of component (a) to component (b) is from 1:6 to 3:1, such as from 1:4 to 3:1 or, particularly, from a lower limit such as 1:3, 2:5 or 1:2 to an upper limit such as 5:2, 2:1 or 3:2 (e.g. a ratio in the range 1:3 to 2:1); and/or
the ratio of the combined mass of components (a) and (b) to the mass of the polysaccharide component (c) is from 1:4 to 4:1, such as from 1:3 to 2:1 or, particularly, from a lower limit such as 2:5, 1:2 or 2:3 to an upper limit such as 3:2, 5:4 or 1:1 (e.g. a ratio in the range 2:5 to 1:1).

Other embodiments of the first aspect of the invention that may be mentioned, which embodiments may be combined with any of the above-mentioned embodiments that are not mutually exclusive, include those in which component (a) forms at least 7.5% by weight of the plastic or gel material (such as at least 10, 12.5, 15 or 20% by weight of said material).

Particular embodiments of the first aspect of the invention relate to a foamed, plastic material comprising components (a), (b) and (c) as described above (and in any of the ratios as described above). The foamed, plastic material may have a density in the range from 0.1 to 1 g/cm³ (e.g. 0.2 to 0.5 g/cm³, such as about 0.3 g/cm³).

A particular embodiment of the first aspect of the invention relates to plastic or gel materials in which:
component (b) is a one or more compounds of formula (IIIa) and/or one or more compounds of formula (IIIb); and
the plastic or gel material is substantially free of polymers that are not polysaccharides (or that are not polysaccharides that are polymers of pyranose monomers, at least 30% of which monomers are in the α-anomeric conformation).

Other embodiments of the invention that may be mentioned relate to plastic or gel materials defined by a combination of this particular embodiment with any one or more embodiments of the first aspect of the invention that are detailed above and that are not mutually exclusive.

In addition to essential components (a), (b) and (c), the plastic or gel material of the first aspect of the invention may contain one or more further additives. Additives that may be mentioned in this respect include those selected from the list comprising fillers, stabilizers, anti-wear agents and blowing agents. Depending upon the nature of the additive, the additive may be incorporated into the plastic or gel material according to the first aspect of the invention either:
by mixing the additive with any one or more of the precursor components of the material (i.e. components (a), (b) and (c)) before those components are converted to the plastic or gel material; or
by mixing the additive with the plastic or gel material after that material has been formed from its precursor components (e.g. using conventional blending techniques).

The precursors employed to form the plastic or gel material of the first aspect of the invention may, in certain embodiments, all be non-toxic to humans. The materials of such embodiments therefore have application in the preparation of (oral) medicaments and foodstuffs.

Thus, in alternative embodiments of the first aspect of the invention, the above-mentioned additive be a foodstuff, a food dye or pharmacologically active agent.

Further, embodiments of the first aspect of the invention that may be mentioned include those in which components (a), (b) and (c) are all:
(i) materials that are non-toxic to humans; and/or
(ii) pharmaceutically acceptable materials.

The terms “non-toxic to humans” and “pharmaceutically acceptable” will be well understood by those skilled in the art. In this respect, the term “non-toxic to humans” includes references to materials that have an LD₅₀ in humans of at least 12.5 mg/kg (such as at least 25, 50, 75 or, particularly, 100 mg/kg). Further, whether or not a material is pharmaceutically acceptable can be determined, for example, by reference to standard texts (such as a pharmacopeia) and/or by routine safety tests.

Alternatively, additional materials may be combined with the plastic or gel material, such as structurally reinforcing materials.

Thus, according to a second aspect of the invention, there is provided a composite material comprising:
(A) a plastic or gel material according to any one of the above-described embodiments of the first aspect of the invention; and
(B) another material.

The other material mentioned at (B) above may be a natural material (e.g. plant-derived material, such plant material employed as a source of polysaccharide component (c)), a metal, an alloy, a ceramic, a glass, and/or a synthetic material such as a plastic, a resin or carbon fibres or nanotubes. In certain embodiments of the second aspect of the invention, the other material is:
(i) a structurally reinforcing material; or
(ii) one or more materials selected from the list comprising cellulose fibres, lignin fibres, carbon fibres, glass fibres or ceramic powders.

The composite material according to the second aspect of the invention may be formed either:
(I) by mixing the other material with any one or more of the precursor components of the plastic or gel material of the first aspect of the invention (i.e. components (a), (b) and (c)) before those components are converted to the plastic or gel material; or
(II) by mixing the other material with the plastic or gel material of the first aspect of the invention after that material has been formed from its precursor components (e.g. using conventional techniques for forming composites of plastics with other materials).

The plastic or gel material according to the first aspect of the invention may be prepared by a process comprising providing a mixture of components (a), (b) and (c), as described above. Thus, according to a third aspect of the invention there is provided a process for preparing a plastic or gel material as defined in the first aspect of the invention, said process comprising:
(i) providing a mixture of
   (a) a compound of formula (I) or a mixture of two or more compounds of formula (I), as defined above,
   (b) one or more uncharged organic compounds, each of which compounds comprises at least one oxygen atom and at least one hydrogen atom that is capable of forming a hydrogen bond with anion Xⁿ⁻ of the compound of formula (I), and
   (c) one or more polysaccharides, wherein each polysaccharide is a polymer of pyranose monomers, at least 30% of which monomers are in the α-anomeric conformation,
   and optionally one or more additives,
   and then heating the mixture until the plastic or gel material is formed; or
(ii) for the preparation of foamed, plastic materials of the first aspect of the invention, providing a mixture of components (a), (b) and (c), and optionally an additive, as defined in (i) above, and a volatile material, and then and then heating the mixture until a plastic material is formed and the volatile material has volatilised and imparted a foamed structure to the plastic material.

Formation of the plastic or gel material according to the first aspect of the invention can be determined, for example, by inspection of the properties of the mixture (of components (a), (b) and (c) and, optionally, an additive) after heating.

As components (a), (b) and (c) are all non-plastic, the conditions (e.g. temperature and duration of heating) required to form the plastic or gel material can easily be determined for any given mixture of such components. However, embodiments of the third aspect of the invention that may be mentioned include those in which the mixture of components (a), (b) and (c) (optionally including an additive) is:
heated to a temperature from 50 to 200° C., such as from 75 to 190° C. or from 100 to 180° C. (e.g. 150° C. or 160° C.);
heated for any length of time from 30 seconds to 240 minutes, such as from 1 to 180 minutes or from 2 to 120 minutes (e.g. from 3 to 30 minutes); and/or
heated under atmospheric pressure or, alternatively, under elevated pressure (e.g. from 200 to 2,000 kPa, such as from 900 to 1,100 kPa).

When both heat and pressure are used to form the material according to the first aspect of the invention, they may both be conveniently applied by way of a heated press.

When used herein in relation to alternative process (b) above, the term “volatile material” includes references to materials that, at atmospheric pressure (e.g. 101.325 kPa) and upon heating to moderate temperatures (e.g. a temperature in the range from 50 to 180° C.), convert from solid or liquid form to entirely gaseous form. In this respect, volatile materials that may be mentioned include volatile organic solvents such as any one or more compounds selected from the list comprising dichloromethane, diethylether, ethanol, ethylacetate, hexane, pentane and acetone.

For the avoidance of doubt, components (a), (b) and (c), as well as the optional additives, may take any of the definitions provided above in relation to the first aspect of the invention.

Further, in the processes of the third aspect of the invention, the ratios of components (a), (b) and (c) may be the same as those described in relation to the plastic or gel material according to the first aspect of the invention.

The components (a), (b) and (c) may be mixed in any order. For example, components (a) and (b) may be mixed together first and then component (c) added to the mixture so formed. As another example, components (a), (b) and (c) may be mixed together simultaneously.

In an alternative embodiment of the third aspect of the invention, component (c) may be replaced by a source of one or more polysaccharides, wherein each polysaccharide is a polymer of pyranose monomers, at least 30% of which monomers are in the α-anomeric conformation.

For example, polysaccharides from natural sources (e.g. plant materials such as grains, tubers and fruits) may be employed in unrefined state to form a plastic or gel material according to the first aspect of the invention, or a composite material according to the second aspect of the invention.

In this alternative embodiment of the third aspect of the invention, the source of one or more polysaccharides, wherein each polysaccharide is a polymer of pyranose monomers, at least 30% of which monomers are in the α-anomeric conformation (e.g. a natural source such as grains, tubers and fruits) is contacted with components (a) and (b) (which components are optionally pre-mixed together) and then heated until a plastic or gel material according to the first aspect of the invention, or a composite material according to the second aspect of the invention, is formed.

In relation to the above, suitable natural sources of polysaccharides that are polymers of pyranose monomers, at least 30% of which monomers are in the α-anomeric conformation include natural sources of agar and starches, such as corn, wheat, tapioca, potato peel, banana peel, orange peel, algae from the genera Gelidium and Gracilaria or the seaweed Sphaerococcus euchema.

When such a natural source of polysaccharide is employed, any residual materials from that natural source (e.g. materials, such as cellulose, that are not polysaccharides that are polymers of pyranose monomers, at least 30% of which monomers are in the α-anomeric conformation) may either be:
retained in the material formed by the process of the alternative embodiment of the third aspect of the invention (thereby providing a composite material according to the second aspect of the invention); or
separated from the plastic or gel material formed from the one or more polysaccharides that are polymers of pyranose monomers, at least 30% of which monomers are in the α-anomeric conformation (thereby providing a plastic or gel material according to the first aspect of the invention).

In another alternative embodiment of the third aspect of the invention, composite materials according to the second aspect of the invention may be formed by providing a mixture of components (a), (b), (c) and another material, and optionally an additive, and then heating that mixture until the composite material according to the second aspect of the invention is formed. In this alternative embodiment, the other material may be, for example a structurally reinforcing material, such as one or more materials selected from the list comprising cellulose fibres, lignin fibres, carbon fibres, glass fibres or ceramic powders.

The gel or plastic materials of the first aspect of the invention, and the composite material of the second aspect of the invention have, due to their mechanical and physicochemical properties, a wide variety of uses, such as for packaging materials. Thus, according to a fourth aspect of the invention, there is provided the use as a packaging material of a plastic or gel material according to the first aspect of the invention, or a composite material according to the second aspect of the invention.

A wide variety of different forms of packaging materials exist (e.g. protective films, rigid casings or soft, foamed materials). However, the foamed, plastic materials according to the above-described particular embodiment of the first aspect of the invention find particular application as packaging materials for protecting delicate or breakable items (e.g. as a replacement for packaging based upon expanded polystyrene).

Because they contain an ionic compound (component (a)) and a medium through which the ions of that compound can migrate (component (b)), plastic or gel materials according to the present invention are capable of conducting an electric current. Thus, according to a fifth aspect of the invention there is provided the use as an electrical conductor of a plastic or gel material according to the first aspect of the invention, or a composite material according to the second aspect of the invention.

Because of the further possibility of other ionic species to migrate through plastic or gel materials according to the present invention (at speeds determined in part by the size of and overall charge of such species), it is possible to use those materials to separate mixtures of ionic species by electrophoresis. Thus, according to a sixth aspect of the invention there is provided the use as a matrix for electrophoresis of a plastic or gel material according to the first aspect of the invention, or a composite material according to the second aspect of the invention. Further, according to an alternative embodiment of the invention, there is provided a method of performing electrophoresis upon one or more ionic species, said method comprising the steps of:
(i) providing a matrix comprising a plastic or gel material according to the first aspect of the invention, or a composite material according to the second aspect of the invention;
(ii) absorbing into said matrix the one or more ionic species; and
(ii) applying an electrical potential to the matrix.

In this aspect of the invention, the one or more ionic species may be a mixture of ionic species that is capable of being analysed (separated on a matrix) by electrophoresis.

As mentioned above, certain plastic or gel materials according to the first aspect of the invention may be non-toxic to humans, and have application in the preparation of (oral) medicaments. Thus, according to a seventh aspect of the invention, there is provided the use of a plastic or gel material in which all of components (a), (b) and (c) are non-toxic to humans (and/or are pharmaceutically acceptable materials) as a component of an oral medicament (e.g. as a diluent, carrier, capsule shell, or coating for a tablet or capsule). In this respect, forms of the oral medicament that may be mentioned include tablets and capsules.

The gel or plastic materials of the first aspect of the invention, and the composite material of the second aspect of the invention can, due to their mechanical properties, be used in the manufacture of various items. Thus, according to an eighth aspect of the invention, there is provided an article comprising a plastic or gel material according to the first aspect of the invention or a composite material according to the second aspect of the invention.

Particular embodiments of the eighth aspect of the invention include those in which the article is a bag (e.g. a carrier bag), a utensil or packaging material (e.g. a tray or a foam chip). Further, when the plastic or gel material comprises components (a), (b) and (c) that are all non-toxic to humans (and/or are all pharmaceutically acceptable materials) articles according to the eighth aspect of the invention that may be mentioned include tablets and capsules.

Articles prepared from the plastic or gel materials of the first aspect of the invention may be provided with a coating (e.g. a hydrophobic coating) in order to improve their resistance to water. Thus according to a ninth aspect of the invention there is provided an article according to the eighth aspect of the invention, wherein the article comprises a plastic or gel material as defined in respect of the first aspect of the invention, or a composite material as defined in respect of the second aspect of the invention, that is coated with a water-resistant (e.g. hydrophobic) material.

Embodiments of this aspect of the invention include articles wherein the coating of water-resistant material covers at least 50% (e.g. at least 60, 70, 80, 90, 95 or 99%, such as 100%) of the exposed surfaces of the plastic, gel or composite material. In this respect, the term “exposed surfaces”, when used herein, includes references to surfaces of the plastic, gel or composite material that, at room temperature and atmospheric pressure, are accessible to (externally-introduced) liquid water or water vapour.

When used herein, the term “water-resistant material” includes references to materials that, at a temperature of 298 K, have a solubility in water of less than 50 ppm (or less than 50 mg/L, such as less than 25, 10, 5 or 1 mg/L). Examples of water-resistant materials that may be mentioned include waxes (e.g. waxes based upon molecules containing at least 20 C-atoms, such as from 20 to 30 C-atoms), hydrophobic polymers (e.g. polyvinylacetate) and polymers such as polyvinylalcohol. Specific waxes that may be mentioned in this respect include those selected from the group consisting of paraffin wax, beeswax, bayberry wax, candelilla wax, caranday wax, castor bean wax, shellac wax, spermaceti wax, sugar cane wax and wool wax (lanolin).

It has been found that the application of pressure to plastic or gel materials of the first aspect of the invention can modify the physical and/or mechanical properties of those materials (e.g. by increasing translucency, elasticity, toughness and/or hardness, and/or by decreasing ultimate tensile strength and/or Young's Modulus of the material). Thus, according to a tenth aspect of the invention there is provided a method of modifying physical and/or mechanical properties of a plastic or gel material according to the first aspect of the invention, said method comprising the step of applying elevated pressure to the plastic or gel material.

In respect of this aspect of the invention, physical properties that may be mentioned include translucency. Further, mechanical properties that may be mentioned include any one or more of elasticity, toughness, hardness, ultimate tensile strength and Young's Modulus. Thus, the method of the tenth aspect of the invention may, in some embodiments, be:
a method of increasing translucency of the material;
a method of increasing elasticity of the material;
a method of increasing toughness of the material;
a method of increasing hardness of the material;
a method of decreasing ultimate tensile strength of the material; and/or
a method of decreasing Young's Modulus of the material.

The properties of translucency, elasticity, toughness, hardness, ultimate tensile strength and Young's Modulus may be determined by using standard methodology known to those skilled in the art.

When used herein in relation to the tenth aspect of the invention, the term “applying elevated pressure” includes references to applying above-atmospheric pressure (i.e. pressure above 101,325 Pa, such as from 2×10⁵ to 1×10⁷ Pa (2 to 100 bar)) to the material. The pressure may, for example, be conveniently applied by placing the plastic or gel material between two parallel metal plates and using elevated pressure to force the plates towards each other.

The materials according to the present invention have the advantages that:
they may be prepared entirely from renewable, natural materials (e.g. materials that do not derive from fossil fuels, such as polysaccharide-containing food waste);
they may be prepared entirely from materials that are non-toxic (e.g. non-toxic to humans);
they may be completely biodegradable/compostable;
they may biodegrade to materials that are harmless to the environment (e.g. to non-toxic materials);
they may by prepared by simple processes (including: energy- and/or material-efficient processes; process involving a small number of steps such as one- or two-step processes; one-pot processes; and/or processes not requiring chemical modifications of a natural polysaccharide constituent of the materials);
they may have mechanical properties that render them suitable for a wide variety of applications;
they may be prepared either directly in the form required (e.g. a form not requiring further processing, such as moulding, laminating or re-casting) or in a form that is easy to manipulate;
they may be recoverable and/or recyclable (e.g. by simple processes such as dissolution/reformation);
they may be stable to heat and/or flame resistant; and/or
they may be electrically conductive.

Without wishing to be bound by theory, the advantageous mechanical properties of the materials according to the present invention are believed to derive from the conversion (by use of components (a) and (b)) of the polysaccharide (component (c)) from its largely crystalline native form to a stable, essentially amorphous form having reduced inter- and/or intra-chain H-bonding.

### BRIEF DESCRIPTION OF THE FIGURES

FIG. 1 is a differential scanning calorimetry (DSC) trace obtained from a sample prepared by mixing choline chloride (component (a)) and glycerol (component (b)) in a 1:2 molar ratio, and then suspending an equal weight (compared to the combined mass of components (a) and (b)) of corn starch in the resulting liquid. The scan rate used in obtaining the DSC trace was 10° C./min and the temperature range covered was from −50 to 400° C.

FIG. 2a is an SEM image for potato starch, showing the granular structure of this material.

FIG. 2b is an SEM image of the sample produced in Example 2 below (a plastic material according to the present invention that was prepared from potato starch). The granular structure of the starch is no longer present once it has been heated with the components (a) and (b) as defined above (which, in the case of Example 2 were choline chloride and urea, respectively).

FIG. 3a shows a suspension prepared by mixing choline chloride (5.36 g) and urea (4.64 g) with corn starch, to which suspension 10% by weight of acetone has been added.

FIG. 3b shows the expanded (foamed) material that is formed after heating the suspension shown in FIG. 3a at 150° C. for 30 minutes.

FIG. 4 shows a sample of the plastic material from Example 1 below (prepared from corn starch, choline chloride and urea by heating the mixture of components at 150° C. in a cylindrical brass mould for 30 minutes) which has been rolled flat to a width of 2.6 mm through callendering.

FIG. 5 shows a 10 cm×10 cm×1 mm sheet of plastic material (comprising corn starch, choline chloride and ethylene glycol) prepared according to an alternative method, namely pressing the mixture of components together at 160° C. and 1 MPa pressure for 3 minutes.

### EXAMPLES

### Examples 1 to 6

Choline chloride (5.36 g, 0.038 mol) was added to urea (4.64 g, 0.077 mol) and a starch (as defined in Table 1) (10 g) and ground with a pestle and mortar. The resulting mixture was then heated to 150° C. in a cylindrical brass mould for 30 minutes.

TABLE 1

| Example | Polysaccharide | Appearance |
|---|---|---|
| 1 | Corn Starch | Flexible solid sample |
| 2 | Potato Starch | Flexible solid sample |
| 3 | Wheat Starch | Flexible solid sample |
| 4 | Tapioca Starch | Flexible solid sample |
| 5 | Agar | Flexible solid sample |
| 6 | Rice Starch | Flexible solid sample |
| 7 | Soluble starch | Flexible solid sample |

### Examples 8 to 12

An amide (as defined in Table 2 below) (2 mole equivalents) was mixed with choline chloride (1 mole equivalent). The resulting mixture was mixed with corn starch (1 weight equivalent relative to the mixture of amide and choline chloride) and resulting mixture heated in an oven at a temperature of 150° C. for 30 minutes.

TABLE 2

| Example | Amide | Appearance |
|---|---|---|
| 8 | Acetamide | Flexible solid sample |
| 9 | Trifluoroacetamide | Weak and crumbly |
| 10 | Benzamide | Flexible solid sample |
| 11 | N-methylurea | Flexible solid sample |
| 12 | N,N′-dimethylurea | Flexible solid sample |

### Examples 13 to 18

A polyol (as defined in Table 2 below) was mixed with choline chloride (molar ratio of polyol:choline chloride defined below). The resulting mixture was added to corn starch (1 weight equivalent relative to the preceding mixture) to form a mixture that was then heated in an oven at a temperature of 150° C. for 30 minutes.

TABLE 3

| Example | Polyol | Molar Ratio | Appearance |
|---|---|---|---|
| 13 | Ethylene glycol | 1:2 | Flexible solid sample |
| 14 | Glycerol | 1:2 | Flexible solid sample |
| 15 | Glycerol | 1:1 | Flexible solid sample |
| 16 | Xylitol | 1:1 | Flexible solid sample |
| 17 | Mannitol | 1:1 | Flexible solid sample |
| 18 | Fructose | 1:1 | Weak and crumbly sample |

### Examples 19 to 22

Urea (component (b); 2 mole equivalents) and choline chloride (component (a); 1 mole equivalent) were mixed with corn starch (weight equivalent relative to the combined mass of components (a) and (b) as defined in Table 4 below) and the resulting mixture heated in an oven at a temperature of 150° C. for 30 minutes.

TABLE 4

| | Ratio of components | |
|---|---|---|
| Example | (a) & (b):corn starch | Appearance |
| 19 | 1:1.2 | Flexible solid sample |
| 20 | 1:1.5 | Strong, flexible solid sample |
| 21 | 1:1.8 | Strong, solid sample |
| 22 | 1:2.3 | Weak, brittle solid |

### Examples 23 to 28

Urea (component (b)) and choline chloride (component (a)) (the molar ratio of component (a) to component (b) being as defined in Table 5 below) were mixed with corn starch (1.2 weight equivalents relative to the combined mass of components (a) and (b)) and resulting mixture heated in an oven at a temperature of 150° C. for 30 minutes.

TABLE 5

| | Molar Ratio of | |
|---|---|---|
| Example | Choline Chloride:Urea | Appearance |
| 23 | 1:3 | Flexible solid sample |
| 24 | 1:1.75 | Flexible solid sample |
| 25 | 1:1.5 | Flexible solid sample |
| 26 | 1:1.25 | Flexible solid sample |
| 27 | 1:1 | Flexible solid sample |
| 28 | 1:0.75 | Flexible solid sample |
| 29 | 1:0.5 | Flexible solid sample |

### Examples 30 to 35

Choline chloride (5.36 g, 0.038 mol) was added to urea (4.64 g, 0.077 mol) and a mixture of corn starch and cellulose (10 g) (ratio by weight of corn starch to cellulose as defined in Table 6 below). The resulting mixture was then heated to 150° C. in a cylindrical brass mould for 30 minutes.

TABLE 6

| Example | Ratio corn starch:cellulose (wt) | Appearance |
|---|---|---|
| 30 | 9:1 | Flexible solid sample |
| 31 | 8:2 | Flexible solid sample |
| 32 | 7:3 | Flexible solid sample |
| 33 | 6:4 | Flexible solid sample |
| 34 | 5:5 | Flexible solid sample |
| 35 | 4:6 | Flexible solid sample |

### Examples 36 to 38

Urea (component (b); 2 mole equivalents) was mixed with an ammonium salt (component (a); 1 mole equivalent; as defined in Table 7 below) and corn starch (1 weight equivalent relative to the combined mass of components (a) and (b)) and the resulting mixture heated in an oven at a temperature of 150° C. for 30 minutes.

TABLE 7

| Example | Ammonium salt | Appearance |
|---|---|---|
| 36 | Acetylcholine chloride | Flexible solid sample |
| 37 | Tetrabutylammonium chloride | Flexible solid sample |
| 38 | Benzyltrimethylammonium chloride | Flexible solid sample |

### Example 38

The irreversible gellation of starch particles through interaction with components (a) and (b) as defined above can be identified by differential scanning calorimetry (DSC).

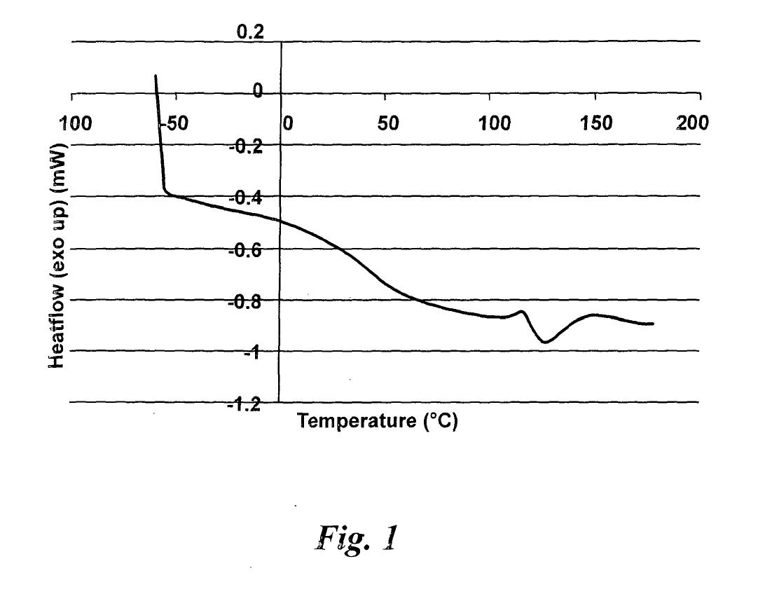

A sample was prepared by mixing choline chloride (component (a)) and glycerol (component (b) in a 1:2 molar ratio. An equal weight (compared to the combined mass of components (a) and (b)) of corn starch was then suspended in the resulting liquid. The sample so prepared was then subjected to differential scanning calorimetry at a scan rate of 10° C./min and from −50 to 400° C. FIG. 1 shows the DSC trace obtained from the sample. As will be evident from the inflection in the heat flow with a midpoint at 46° C., an irreversible gellation of the sample occurred between 20 and 80° C.

### Example 39

Appearance of Samples

Starch has a granular structure with particles between 1 and 40 μm in diameter.

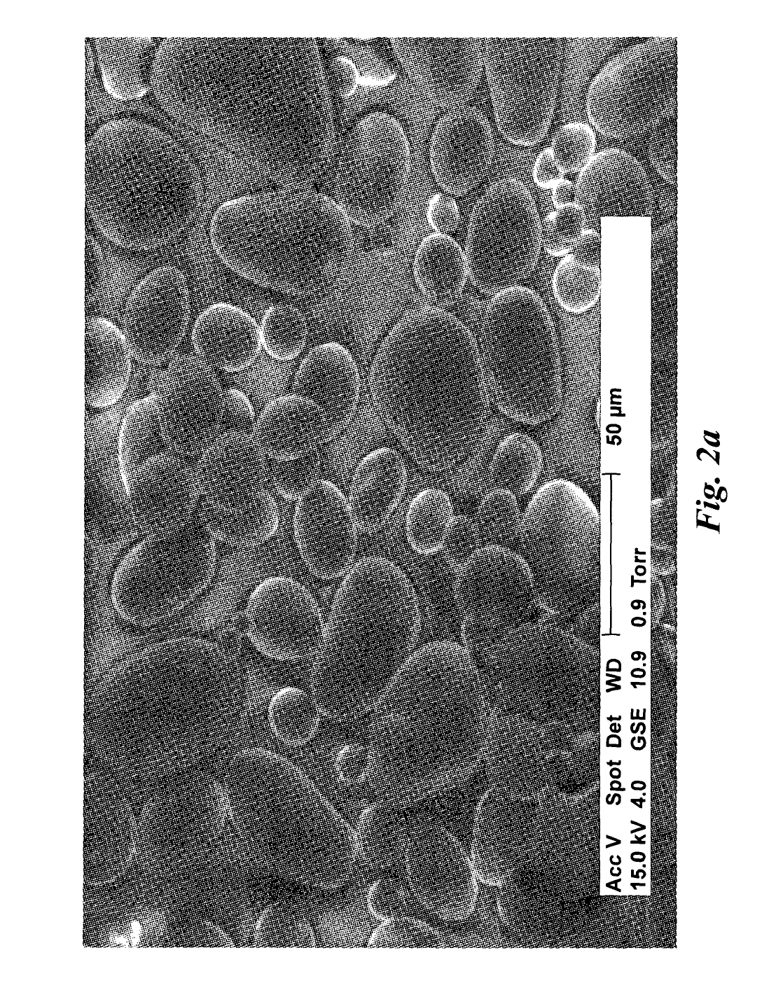

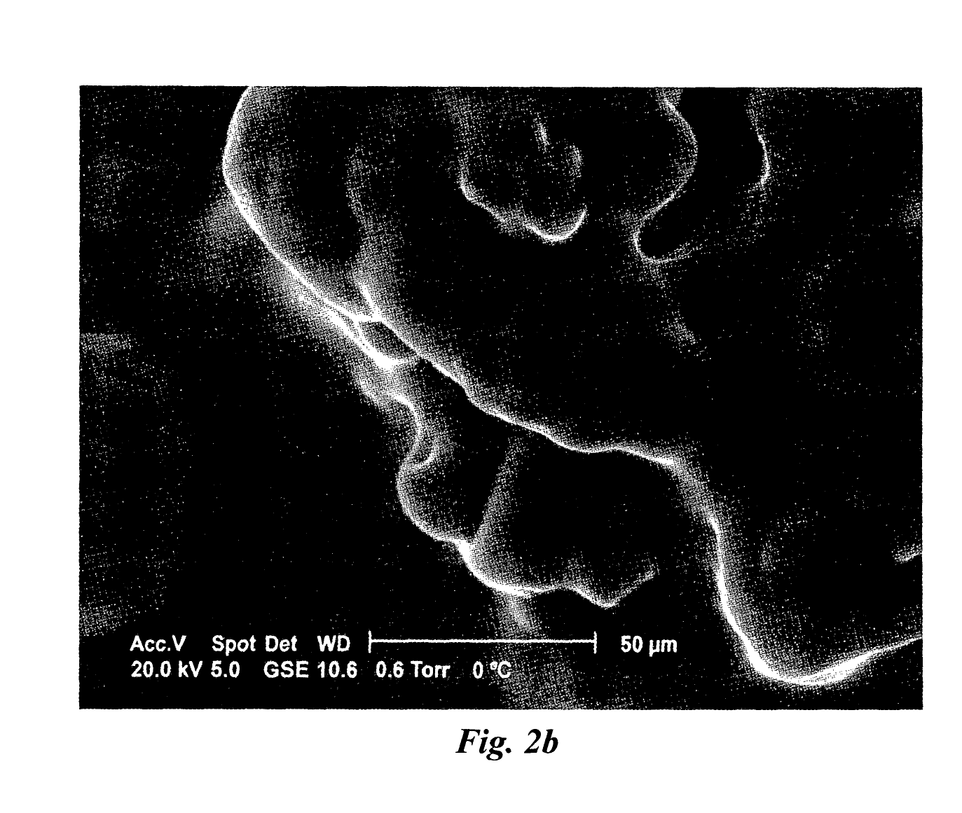

FIG. 2a shows the SEM image for potato starch, showing the granular structure of this material. By way of contrast, FIG. 2b shows the SEM image of the sample produced in Example 2. The granular structure of the starch is no longer present once it has been heated with the components (a) and (b) as defined above (which, in the case of Example 2, were choline chloride and urea, respectively). Similar SEM results have been observed for Examples 1-6.

### Example 40

Mechanical Properties of Modified Starch

Known diameter samples of materials produced according to preceding examples were subjected to stress/strain analysis using a strain gauge at 298 K. From the stress/strain curves produced, the ultimate tensile strength (UTS) and elongation at break (% EL) were determined for the samples, and are reported in Table 8 below.

TABLE 8

| Example | UTS (kN m⁻²) | % EL |
|---|---|---|
| 4 | 44 | 285 |
| 8 | 60 | 78 |
| 19 | 97 | 68 |
| 20 | 394 | 20 |
| 24 | 120 | 69 |
| 32 | 107 | 41 |

### Example 41

Conductivity

Because they contain an ionic compound (component (a)) and a medium through which the ions of that compound can migrate (component (b)), plastic or gel materials according to the present invention are capable of conducting an electric current. Thus, for example, the conductivity of the material formed in Example 1 was determined by electrochemical impedance spectroscopy to be 2.18×10⁻⁷ S cm⁻¹. The thickness of the sample was calculated by sandwiching the material between two 2 cm×4 cm nickel plates and measuring using a micrometer.

### Example 42

Foam Packaging

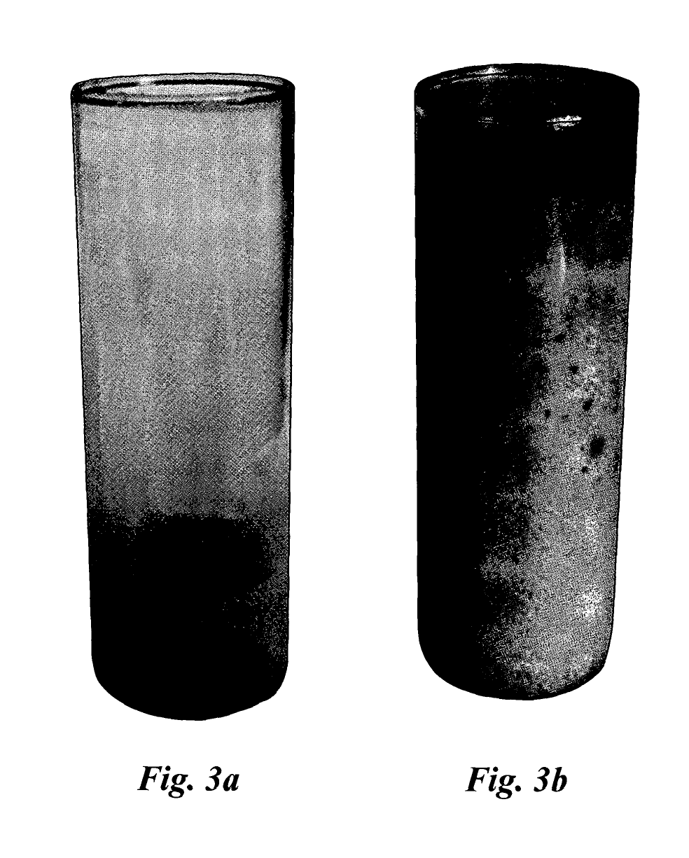

Example 1 above was repeated with the addition of 10% by weight of acetone prior to heating. On heating, the liquid evaporated, causing the formation of gas pockets. After curing and hardening of the product, a solid foam of low density (0.3 g cm⁻³) was obtained. FIG. 3a shows the suspension employed in Example 1 with 10% wt acetone prior to heating. The expanded material formed after heating at 150° C. for 30 minutes is shown in FIG. 3b.

### Example 43

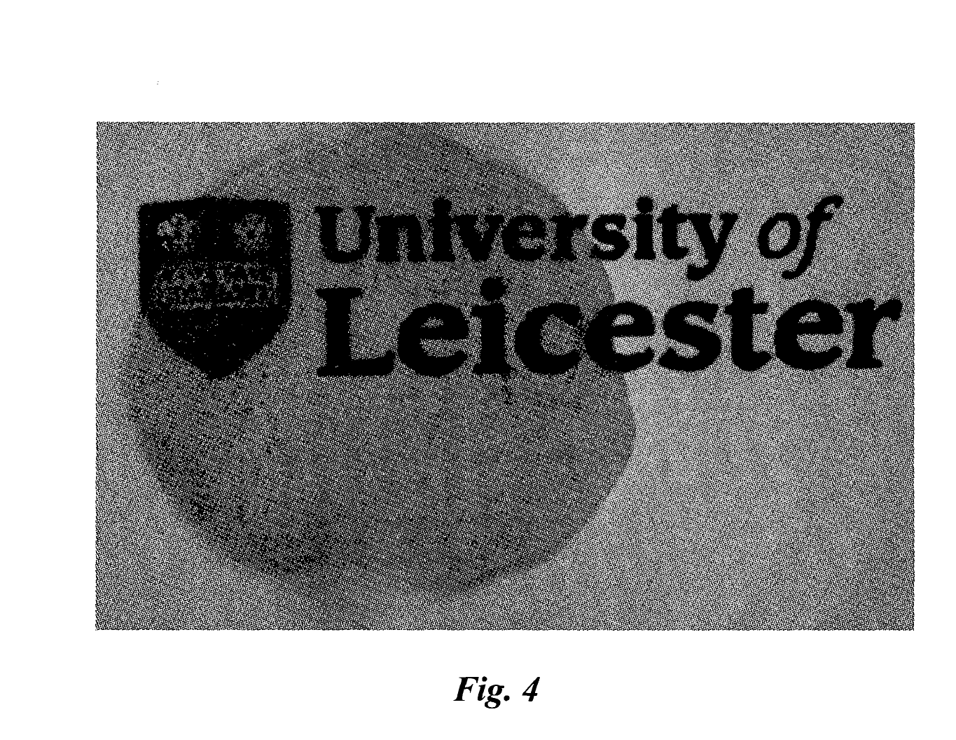

Due to the simplistic nature of the formation of the plastic or gel materials according to the present invention, they can be formed in a variety of different morphologies. In addition once these materials have been formed their shape can be modified through the application of pressure (or by moulding). FIG. 4 shows the material from Example 1 which has been rolled flat to a width of 2.6 mm through callendering.

### Example 44

Electrophoresis

An aqueous solution of methylene blue (blue cationic dye) and potassium hexacyanoferrate (yellow anionic dye) was placed on top of the sample from Example 1 which had been cast as a thin film and allowed to evaporate. An electrical potential of 20 volts was applied using a DC (direct current) power supply to opposite ends of the sample. After a period of 24 hours, the colours were visibly shown to separate, showing a sequence of blue (from methylene blue), green (from overlapping spots) and yellow (from the hexacyanoferrate).

### Example 45

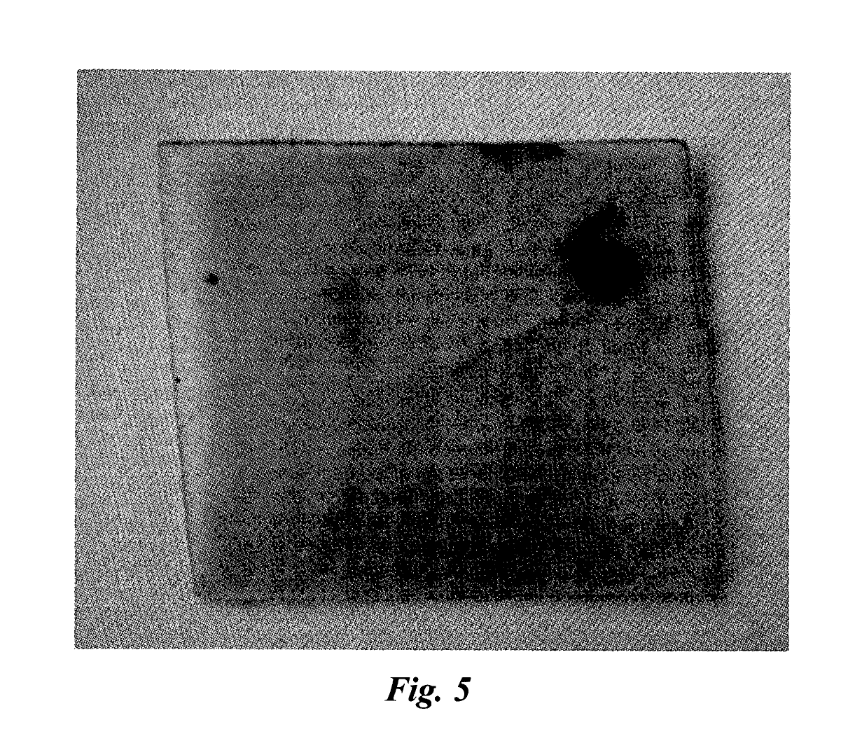

Plastic or gel materials according to the present invention can be produced through the combined use of heat and pressure. Corn starch (10 g), choline chloride (2.27 g) and ethylene glycol (2.01 g) were mixed together and pressed into a sheet having dimensions 10 cm×10 cm×1 mm at 160° C. and 10 bar (1 MPa) pressure for 3 minutes. The resulting film is shown in FIG. 5 and was found to be soft and flexible, although non-conducting.

### Example 46

Maltitol (7.11 g) and choline chloride (2.89 g) were mixed together in a 1:1 molar ratio. The mixture was then heated to 120° C. before being allowed to cool. The resulting mixture was observed to be a solid having a melting point in excess of 100° C. To 10 g of this mixture at 120° C. was added corn starch in a 1:1 ratio by weight. The resulting mixture was ground at high temperature and then put in a cylindrical brass mould for 30 minutes at 150° C. After heating, the mixture eventually solidified upon cooling. The sample removed from the mould gradually lost its shape.

1. A plastic or gel material comprising a mixture of:
(a) a compound of formula (I) or a mixture of two or more compounds of formula (I),
(R⁺)ₙ(Xⁿ⁻) (I)
or a hydrate thereof, wherein
n is 1, 2 or 3,
R⁺ is a primary, secondary, tertiary, quaternary or unsubstituted ammonium cation or a quaternary phosphonium cation,
Xⁿ⁻ is a monovalent, bivalent or trivalent anion;
(b) one or more compounds of formula (IIIa), one or more compounds of formula (IIIb), or a mixture of one or more compounds of formula (IIIa) and one or more compounds of formula (IIIb): 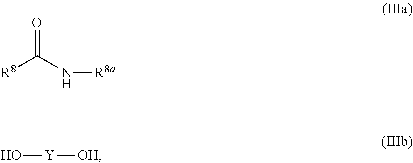
wherein:
R⁸ represents H, C₁₋₄ alkyl which latter group is optionally substituted by one or more F atoms, phenyl which latter group is optionally substituted by one or more substituents selected from halo, C₁₋₄ alkyl and C₁₋₄ alkoxy, or N(R⁹)R¹⁰,
R^{8a} represents H or C₁₋₄ alkyl which latter group is optionally substituted by one or more F atoms,
R⁹ and R¹⁰ independently represent H or C₁₋₄ alkyl which latter group is optionally substituted by one or more F atoms,
Y represents C₂₋₁₀ alkylene or C₄₋₈ cycloalkylene optionally
(i) substituted by one or more substituents selected from F, OH, SH, N(R¹¹)R¹² and C₁₋₄ alkyl which latter group is optionally substituted by one or more substituents selected from F and OH,
(ii) interrupted by one or more groups selected from O, S and NR¹³, or
(iii) both (i) and (ii); and
R¹¹ to R¹³ independently represent H or C₁₋₄ alkyl which latter group is optionally substituted by one or more substituents selected from F and OH; and
(c) one or more polysaccharides, wherein each polysaccharide is a polymer of pyranose monomers, at least 30% of which monomers are in the α-anomeric conformation,
and wherein the plastic or gel material is substantially free of polymers that are not polysaccharides.

2. The plastic or gel material as claimed in claim 1, wherein M⁺ is a quaternary phosphonium cation or a primary, secondary, tertiary, quaternary or unsubstituted ammonium cation.

3. The plastic or gel material as claimed in claim 2, wherein the compound of formula (I) is a compound of formula (Ia),
(R⁺)(X⁻) (Ia)
wherein:
R represents Het substituted by R⁵, or R represents a structure represented by formula (II) 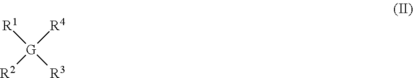
in which G represents N or P;
X⁻ is a monovalent anion;
R¹ to R⁵ independently represent
phenyl, C₁₋₁₀ alkyl, C₂₋₁₀ alkenyl or C₂₋₁₀ alkynyl, which latter four groups are optionally substituted by one or more R⁶ groups, or
C₃₋₁₀ cycloalkyl or C₄₋₁₀ cycloalkenyl which latter four groups are optionally substituted by one or more substituents selected from R⁶ and oxo,
or R⁵ represents H or, when G represents N, R¹ to R⁴ may also independently represent H;
Het represents a 4- to 14-membered heterocyclic group containing a N-atom that is bonded to the substituent R⁵, which heterocyclic group
(i) optionally contains one or more further heteroatoms selected from O, N and S,
(ii) may be fully saturated, partially unsaturated or aromatic,
(iii) may comprise one, two or three rings and
(iv) may be substituted by one or more further substituents selected from R⁶ and oxo;
R⁶ represents, independently at each occurrence,
halo,
CN,
C₁₋₁₀ alkyl, C₂₋₁₀ alkenyl, C₂₋₁₀ alkynyl, C₃₋₁₀ cycloalkyl, C₄₋₁₀ cycloalkenyl, which latter five groups are optionally substituted by one or more substituents selected from halo, C₁₋₄ alkyl, phenyl optionally substituted by one or more substituents selected from OH, halo, C₁₋₄ alkyl and C₁₋₄ alkoxy, and OR^{6a},
OR^{6b},
SR^{6c},
N(R^{6d})(R^{6e}),
phenyl, which latter group is optionally substituted by one or more substituents selected from OH, halo, C₁₋₄ alkyl and C₁₋₄ alkoxy, or
B¹-A-B²—R^{6f};
R^{6a} to R^{6f} independently represent, at each occurrence,
H,
C₁₋₁₀ alkyl, C₂₋₁₀ alkenyl, C₂₋₁₀ alkynyl, C₃₋₁₀ cycloalkyl, C₄₋₁₀ cycloalkenyl, which latter five groups are optionally substituted by one or more substituents selected from halo, C₁₋₄ alkyl, phenyl optionally substituted by one or more substituents selected from OH, halo, C₁₋₄ alkyl and C₁₋₄ alkoxy, OH and C₁₋₄ alkoxy, or
phenyl optionally substituted by one or more substituents selected from OH, halo, C₁₋₄ alkyl and C₁₋₄ alkoxy;
A represents C(O) or S(O)₂;
B¹ and B² independently represent a direct bond, O, S, or N(R⁷); and
R⁷ represents H, C₁₋₄ alkyl, C₃₋₇ cycloalkyl or phenyl, which latter group is optionally substituted by one or more substituents selected from OH, halo, C₁₋₄ alkyl and C₁₋₄ alkoxy.

4. The plastic or gel material as claimed in claim 3, wherein the compound of formula (I) is selected from the list comprising benzyltrimethylammonium chloride, tetrabutylammonium chloride, ethylmethylimidazolium chloride, acetylcholine chloride and choline chloride.

5. The plastic or gel material as claimed in claim 1, wherein the plastic or gel material comprises at most 5% by weight of polymers that are not polysaccharides.

6. The plastic or gel material as claimed in claim 1, wherein the plastic or gel material comprises at most 5% by weight of water.

7. The plastic or gel material as claimed in claim 1, wherein component (b) is one or more compounds selected from the list comprising benzamide, acetamide, N-methylurea, N,N′-dimethylurea urea, glycerol, mannitol, xylitol, ethylene glycol and propylene glycol.

8. The plastic or gel material as claimed in claim 1, wherein the or each polysaccharide of component (c) comprises one or more of amylose, amylopectin, agarose and agaropectin.

9. The plastic or gel material as claimed in claim 8, wherein component (c) is one or more polysaccharides selected from the list comprising agar and a starch.

10. The plastic or gel material as claimed in claim 1, wherein the material comprises a mixture of:
(a) one or more compounds selected from the list comprising benzyltrimethylammonium chloride, tetrabutylammonium chloride, ethylmethylimidazolium chloride, acetylcholine chloride and choline chloride;
(b) one or more compounds selected from the list comprising benzamide, acetamide, N-methylurea, N,N′-dimethylurea urea, glycerol, mannitol, xylitol and propylene glycol; and
(c) one or more polysaccharides selected from the list comprising agar and a starch.

11. A plastic material as claimed in claim 1 that is foamed.

12. The plastic or gel material as claimed in claim 1, wherein the material further comprises an additive selected from the list comprising fillers, stabilizers, anti-wear agents, blowing agents, foodstuffs, food dyes and pharmacologically active agents.

13. A composite material comprising:
(A) plastic or gel material as defined in claim 1; and
(B) another material.

14. The composite material as claimed in claim 13, wherein the other material is selected from the list comprising cellulose fibres, lignin fibres, carbon fibres, glass fibres or ceramic powders.

15. A process for preparing a plastic or gel material as defined in claim 1, said process comprising:
(i) providing a mixture of
(a) a compound of formula (I) or a mixture of two or more compounds of formula (I),
(b) a compound of formula (IIIa), formula (IIIb), or a mixture thereof; and
(c) one or more polysaccharides,
and optionally one or more additives,
and then heating the mixture until the plastic or gel material is formed; or
(ii) for the preparation of foamed, plastic materials, providing a mixture of components (a), (b) and (c), and optionally an additive, as defined in (i) above,
and a volatile material, and then heating the mixture until a plastic material is formed and the volatile material has volatilised and imparted a foamed structure to the plastic material.

16. The process as claimed in claim 15, wherein the mixture is heated to a temperature from 100 to 180° C. for a duration of from 2 to 120 minutes.

17. The plastic or gel material as claimed in claim 1, wherein the molar ratio of component (a) to component (b) is from 1:4 to 3:1.

18. The plastic or gel material as claimed in claim 1, wherein the ratio of the combined mass of components (a) and (b) to the mass of the polysaccharide component (c) is from 1:3 to 2:1.

19. The process as claimed in claim 15, wherein the volatile material is any one or more compounds selected from the list comprising dichloromethane, diethylether, ethanol, ethylacetate, hexane, pentane and acetone.

20. (canceled)

21. An article comprising a plastic or gel material as defined in claim 1.

22. The article as claimed in claim 21, wherein the article comprises a plastic or gel material coated with a water-resistant material.

23. The article as claimed in claim 22, wherein the water-resistant material is polyvinylacetate, polyvinylalcohol or a wax selected from the group consisting of paraffin wax, beeswax, bayberry wax, candelilla wax, caranday wax, castor bean wax, shellac wax, spermaceti wax, sugar cane wax and wool wax (lanolin).

24. The plastic or gel material as claimed in claim 1, wherein, if present, the one or more compounds of formula (IIIb) are one or more compounds of formula (IV), 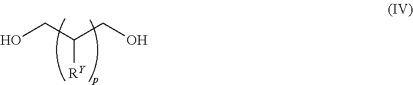
wherein:
R^{Y} is H or OH; and
p is 0 to 4.

25. The article of claim 21, wherein the article is a packaging material, an electrical conductor, or a matrix for electrophoresis.
